(19) United States
(12) Patent Application Publication
(10) Pub. No.: US 2016/0176780 A9
(43) Pub. Date: Jun. 23, 2016

(54) EXCESS AIR CONTROL FOR CRACKER FURNACE BURNERS

(71) Applicant: DOW GLOBAL TECHNOLOGIES LLC, Midland, MI (US)

(72) Inventors: James D. Tate, Lake Jackson, TX (US); Gerald D. Frederick, Sherwood Park (CA); Sylvester Irving, Lake Jackson, TX (US); Charles W. Lipp, Lake Jackson, TX (US); Andy E. Weber, Lake Jackson, TX (US); Chris Reed, Lake Jackson, TX (US)

(21) Appl. No.: 14/248,585

(22) Filed: Apr. 9, 2014

Related U.S. Application Data
(63) Continuation-in-part of application No. 12/735,360, filed on Dec. 1, 2010, now abandoned.
(60) Provisional application No. 60/681,549, filed on May 16, 2005.

(51) Int. Cl. C07C 4/04

(52) U.S. Cl. CPC C07C 4/04

(57) ABSTRACT

A method for control of the air/fuel ratio of the burner(s) (excess air) of a thermal cracker includes three steps. The thermal cracker has three consecutive zones or portions through which combustion gases pass, a firebox portion, a bridge wall portion and a convection portion The first step is to direct a wavelength modulated beam of near infrared light from two different tunable diode lasers located in the bridge wall portion through combustion gas from the burner to a pair of near infrared light detectors, each positioned to receive the wavelength modulated beam of near infrared light from a different one of the two tunable diode lasers to generate a detector signal. The second step is to analyze the detector signals for spectroscopic absorption at wavelengths characteristic of oxygen and carbon monoxide to determine their respective concentrations in the combustion gas. The third step is to adjust the air/fuel ratio of the burner(s) (excess air) in response to the concentrations of oxygen and carbon monoxide of the second step.

### BACKGROUND OF THE INVENTION

The instant invention is in the field of methods for the control of excess air in cracker furnace burners. The production of olefins by thermally cracking a hydrocarbon material, such as petroleum naphtha, is one of the most important processes in the chemical process industry. For example, ABB Corporation reportedly constructed a cracking plant in Port Arthur Texas having a capacity to produce over a million tons of ethylene and propylene per year. The cracking process is conducted in a “cracker”. A cracker usually comprises an enclosure containing tubes and a burner. Heat generated by burning a fuel heats the hydrocarbon material flowing in the tubes so that the hydrocarbon material is thermally cracked to produce, among other things, ethylene and propylene.

Ordinarily, a cracker is comprised of a radiant section and a convection section. The burner is positioned in the radiant section so that the tubes positioned in the radiant section are heated primarily by radiant heat emitted from the walls adjacent to the burner. The combustion gas from the radiant section is then directed to the convection section where heat from the combustion gas is recovered to heat tubes positioned in the convection section. An oxygen sensor, such as a zirconium oxide oxygen sensor, is ordinarily positioned in the cracker between the radiant section and the convection section to facilitate control of the air/fuel ratio of the burner. The overall efficiency of the cracker is primarily a function of the amount of excess air present in the firebox and the temperature of the exhaust gas from the cracker. It can be beneficial from an efficiency viewpoint to control the amount of air in the furnace. Carbon monoxide and smoke emissions from the cracker tend to increase when the amount of air used in the burner is reduced below the stoichiometric ratio of air-to-fuel. On the other hand, too much excess air can reduce the overall efficiency of the cracker and can result in excessive emissions of oxides of nitrogen. Therefore, accurate control of the amount of excess air used in the cracker furnace is necessary for an optimum balancing of efficiency and for the control of emissions.

The oxygen sensor of a conventional cracker furnace is a “point measurement device”, i.e., it measures oxygen corresponding to a small volume at the position where the sensor is located. Such a measurement is not representative of the oxygen concentration in the cracker furnace as a whole. It would be an advance in the art of the control of cracker furnaces if a system were developed that provided a more representative determination of oxygen in the cracker. Also, it is well known that conventional zirconium oxide sensors are subject to interferences known to affect the accuracy of the O₂ measurement (such as hydrocarbons and CO gases). It would be an advance in the art of the control of cracker furnaces if a system were developed that was more immune to these interferences.

Section II.4.3, Sensors for Advanced Combustion Systems, Global Climate & Energy Project, Stanford University, 2004, by Hanson et al., summarized the development of the tunable near-infrared diode laser and absorption spectroscopy approach for the determination of oxygen, carbon monoxide and oxides of nitrogen in the combustion gas from a coal fired utility boiler, a waste incinerator as well as from jet engines. Thompson et al., U.S. Patent Application Publication U.S. 2004/0191712 A1 applied such a system to combustion applications in the steelmaking industry. It would be an advance in the art if the tunable near-infrared diode laser and absorption spectroscopy approach for the determination of oxygen, carbon monoxide and oxides of nitrogen in combustion gas were applied to thermal crackers.

### SUMMARY OF THE INVENTION

The instant invention is a solution, at least in part, to the above-stated problem of the need for a more reliable and representative analysis of combustion gas from a thermal cracker furnace. The instant invention is the application of the tunable near-infrared diode laser and absorption spectroscopy approach for the determination of, for example, oxygen, carbon monoxide and oxides of nitrogen in the combustion gas from a thermal cracker furnace.

More specifically, the instant invention is a method for control of the air/fuel ratio of the burners of a thermal cracker for producing olefins which comprises a firebox portion, a bridge wall portion and a convection portion, comprising the steps of: (a) directing a wavelength modulated beam of near infrared light from two tunable diode lasers that are positioned with a line of sight through combustion gas from burners located in the firebox portion at a location in the bridge wall portion where mixing of the combustion gas is uniform, one of the tunable diode lasers being tuned to a frequency characteristic of oxygen to establish a signal for oxygen content of the combustion gas and one being tuned to a frequency characteristic of carbon monoxide to establish a signal for carbon monoxide content of the combustion gas to a pair of near infrared light detectors, one for each tunable diode laser, to generate two detector signals, one for each of oxygen and carbon monoxide; (b) analyzing the detector signals for spectroscopic absorption at wavelengths characteristic of oxygen and carbon to determine their respective concentration in the combustion gas; and (c) adjusting the air/fuel ratio of the burners (i.e. excess air in the furnace) in response to the concentrations of the oxygen and carbon monoxide of step (b).

As used herein, each of “uniform mixing” and “a location where mixing is uniform” equates to a location that does not include a recirculation zone. A preferred placement of the two tunable diode lasers locates them such that their line of sight focuses upon a location where mixing is uniform. The location preferably provides conditions consistent with those in the combustion zone such that gas concentrations for oxygen and carbon monoxide at the location represent or indicate a true air-to-fuel ratio present in the combustion zone proximate to burners contained in the combustion zone.

The method of this invention employs two tunable diode lasers (TDLs), one for each of oxygen and carbon monoxide. Skilled artisans recognize that current equipment limitations of TDLs provide some ability to vary frequency, but not enough that a single TDL can be tuned to cover frequencies as disparate as those for carbon monoxide (wavelength of number of 2325 nanometers (nm) to 2330 nm) and oxygen (wavelength of 760 nm to 764 nm). If desired, one can add one or more TDLs to measure other combustion gases such as nitrogen oxides, but doing so increases costs associated with measurement and does not provide a concurrent increase in speed or accuracy of measuring carbon monoxide and oxygen.

The two TDLs may be positioned such that they are parallel to one another or orthogonal to each other or canted such that their beams cross one another so long as their respective beams intersect a location in the bridge wall portion of the thermal cracker where mixing is uniform and thereafter enter into operative contact with an associated near infrared light detector (i.e. each TDL is paired with a near infrared light detector). Beams from the two TDLs pass directly through combustion gases at the above location without previously passing through a multiplexer or thereafter passing through a demultiplexer.

### BRIEF DESCRIPTION OF THE DRAWINGS

FIG. 1 is a schematic side view of a typical thermal cracking furnace 10 for producing olefins;

FIG. 2 is a schematic rear view of the furnace 10 of FIG. 1 schematic rear view of the furnace 10 of FIG. 1;

FIG. 3 is a detailed view of a preferred tunable diode laser spectroscopy apparatus for use in the instant invention;

FIG. 4 is a spectrum collected using the system of the instant invention showing fine structure absorbance in the wavelength region characteristic for oxygen absorbance of near infrared light generated by a tunable diode laser.

### DETAILED DESCRIPTION

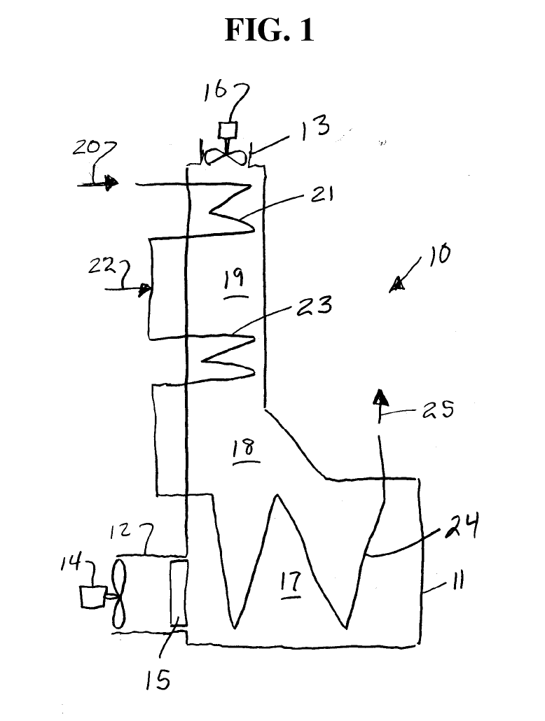

FIG. 1 shows a schematic side view of a typical thermal cracking furnace 10 for producing olefins including an enclosure 11 having an air inlet 12 and an exhaust outlet 13. An air inlet fan 14 provides forced draft through a burner 15. An exhaust fan 16 provides an induced draft from the furnace 10. The interior of the furnace 10 is comprised of three primary portions: the firebox portion 17; the bridge wall portion 18; and the convection portion 19. Combustion gases from the burner 15 are first directed into the firebox portion 17 of the furnace 10, then through the bridge wall portion 18, then through the convection portion 19 and then out of the exhaust outlet 13. Feed stream 20 is conducted through tubing 21 to preheat the feed. Steam 22 is introduced to the preheated feed which is then further heated by tubing 23 positioned in the convection portion 19 and then further heated by tubing 24 positioned in the firebox portion 17 to produce a product 25.

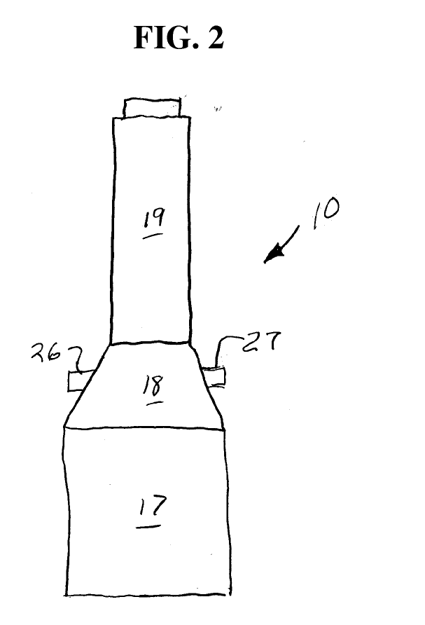

Referring now to FIG. 2, therein is shown a schematic rear view of the furnace 10 of FIG. 1 showing the exterior walls of the firebox portion 17, the bridge wall portion 18 and the convection portion 19. A tunable diode laser system 26 is mounted at the bridge wall portion 18 of the furnace 10 so that light from the tunable diode laser of the tunable diode laser system 26 can be shown through the combustion gas flowing through the bridge wall portion 18 to a light detector system 27.

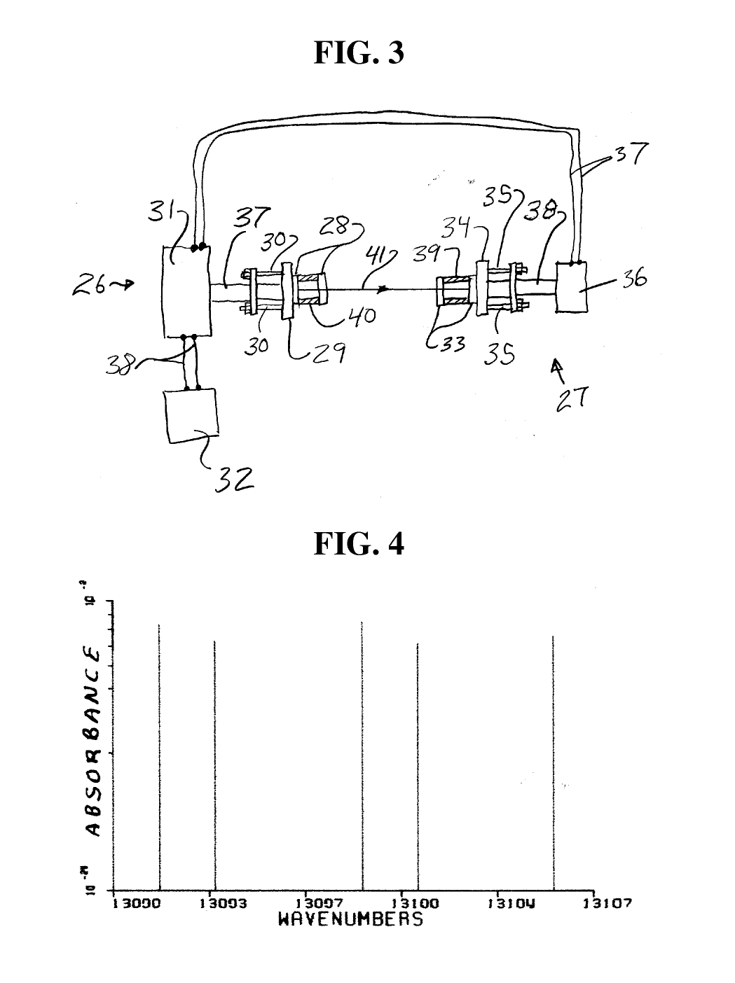

Referring now to FIG. 3, therein is shown a more detailed view of the diode laser system 26 and light detector system 27 shown in FIG. 2. The system shown in FIG. 3 includes a laser module 37 containing the tunable diode laser. A control unit 31 contains the central processing unit programmed for signal processing (to be discussed below in greater detail) as well as the temperature and current control for the tunable diode laser and a user interface and display. The control unit may be contained in a separate unit as shown or may be included in one of the other components of the system, e.g. control unit contained in the transmitter. Alignment plate 29 and adjustment rods 30 allow alignment of the laser beam 41. The laser beam passes through a window or windows (e.g. fused silica windows, sapphire windows) into the furnace. The windows, such as dual sapphire windows 28 may be mounted in a four inch pipe flange 40. The space between the windows 28 is purged with 25 Liters per minute of nitrogen at ten pounds per square inch gauge pressure. The flange 40 is mounted through the wall of the furnace.

Referring still to FIG. 3, the laser beam 41 is passed through a window or windows 33 (they may be dual sapphire or other suitable material such as fused silica) to a near infrared light detector 38. The windows 33 may be mounted in a four inch pipe flange 39. The space between the windows 33 is purged with 25 Liters per minute of nitrogen at ten pounds per square inch gauge pressure. The flange 39 is mounted through the wall of the furnace. Alignment plate 34 and adjustment rods 35 allow alignment of the detector optics with the laser beam 41. Detector electronics 36 are in electrical communication with the control unit 31 by way of cable 37. The control unit 31 is also in electrical communication with the process control system 32 for controlling the furnace 10 (by way of electrical cables 38). The optical path length of the laser beam 41 is about sixty feet. The system shown in FIG. 3 is commercially available from Analytical Specialties of Houston, Tex.

The system shown in FIG. 3 operates by measuring the amount of laser light that is absorbed (lost) as it travels through the combustion gas. Oxygen, carbon monoxide and nitrogen oxide each have spectral absorption that exhibits unique fine structure. The individual features of the spectra are seen at the high resolution of the tunable diode laser 37. The tunable diode laser 37 is modulated (that is scanned or tuned from one wavelength to another) by controlling its input current from the control unit 31.

Referring now to FIG. 4, therein is shown a spectrum in the region where oxygen absorbs the modulated beam of near infrared light from the tunable diode laser. The absorbance shown in FIG. 4 is proportional to the concentration of oxygen in the combustion gas. A carbon monoxide absorbance line near 2333 nanometers is used to determine low parts per million concentration of carbon monoxide. A carbon monoxide absorbance line near 1570 is used to determine higher concentrations of carbon monoxide. A nitrogen oxide absorbance line near 2740 nanometers is used to determine low to sub parts per million concentration of nitrogen oxide. A nitrogen oxide absorbance line near 1800 is used to determine higher concentrations of nitrogen oxide.

Referring again to FIG. 1, the air/fuel ratio of the burners (excess air in furnace) 15 (which is controlled by the process controller 32 of FIG. 3) can be controlled to optimize the oxygen, carbon monoxide and nitrogen oxide concentrations in the combustion gas in response to the tunable diode laser spectroscopic analysis of oxygen, carbon monoxide and nitrogen oxide outlined above.

### CONCLUSION

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

What is claimed is:

1. A method for control of the air/fuel ratio of the burner(s) of a thermal cracker for producing olefins which comprises a firebox portion, a bridge wall portion and a convection portion, comprising the steps of: (a) directing a wavelength modulated beam of near infrared light from two tunable diode lasers that are positioned with a line of sight through combustion gas from burners located in the firebox portion at a location in the bridge wall portion where mixing of the combustion gas is uniform, one of the tunable diode lasers being tuned to a frequency characteristic of oxygen to establish a signal for oxygen content of the combustion gas and one being tuned to a frequency characteristic of carbon monoxide to establish a signal for carbon monoxide content of the combustion gas, to a pair of near infrared light detector to generate two detector signals, one for each of oxygen and carbon monoxide; (b) analyzing the detector signals for spectroscopic absorption at wavelengths characteristic for oxygen and carbon monoxide to determine their respective concentration in the combustion gas; and (c) adjusting the air/fuel ratio of the burners (excess air) in response to the concentrations of oxygen and carbon monoxide of step (b).

2. The method of claim 1, wherein the wavelength used to determine concentration of oxygen is within a range of from 760 nanometers to 764 nanometers.

3. The method of claim 1 wherein the wavelength used to determine concentration of carbon monoxide is within a range of from 2325 nanometers to 2330 nanometers.
